(19) United States
(12) Patent Application Publication
(10) Pub. No.: US 2011/0218151 A1
(43) Pub. Date: Sep. 8, 2011

(54) SUBSTANCES FOR PROMOTING HEALTHY JOINT FUNCTION COMPRISING OMEGA-3 POLYUNSATURATED FATTY ACIDS OR DRIVATIVES THEREOF, UNDENATURED TYPE II COLLAGEN AND, OPTIONALLY, GLUCOSAMINE SULFATE

(76) Inventor: Joar Opheim, Aptos, CA (US)

(21) Appl. No.: 13/038,155

(22) Filed: Mar. 1, 2011

Related U.S. Application Data
(63) Continuation-in-part of application No. 12/717,787, filed on Mar. 4, 2010.

(51) Int. Cl. A61K 38/39; A61P 19/02

(52) U.S. Cl. 514/17.2

(57) ABSTRACT

Medicaments and therapeutic compositions contain (1) at least one omega-3 polyunsaturated fatty acid, or at least one pharmaceutically acceptable omega-3 polyunsaturated fatty acid derivative or mixtures thereof, (2) undenatured Type II collagen and, optionally, (3) glucosamine sulfate. One source of the fatty acids or derivatives thereof is fish oil. The compositions are useful for promoting joint health in a subject.

### CROSS-REFERENCE TO RELATED APPLICATION

This application is a continuation-in-part of U.S. patent application Ser. No. 12/717,787, filed Mar. 4, 2010, commonly owned, the contents of which are hereby incorporated by reference.

### BACKGROUND OF THE INVENTION

The present invention relates to compositions employed in therapeutic compositions, nutritional supplements, and/or medicaments wherein the compositions are combinations comprising (1) omega-3 polyunsaturated fatty acid, a derivative of omega-3 polyunsaturated fatty acid or mixtures thereof, (2) undenatured Type II collagen (sometimes called “UC-II”), and, optionally, (3) glucosamine sulfate. The omega-3 polyunsaturated fatty acids or derivatives thereof can be derived from fish oils or other sources of omega-3 polyunsaturated fatty acids or derivatives thereof. In some embodiments, the invention relates to methods of promoting joint health in a subject by administering to the subject a composition of the invention.

### DESCRIPTION OF THE PRIOR ART

U.S. Pat. No. 5,529,786, issued Jun. 25, 1996 to Moore; U.S. Pat. No. 5,637,321, issued Jun. 10, 1997 to Moore; U.S. Pat. No. 5,645,851, issued Jul. 8, 1997 to Moore; and U.S. Pat. No. 5,750,144, issued to Moore, each disclose undenatured Type II collagen and its use in alleviating the symptoms of arthritis in mammals. Each of these patents is incorporated by reference herein in its entirety.

Gruenwald, J. et al., “Effect of Glucosamine Sulfate with or without Omega-3 Fatty Acids in Patients with Osteoarthritis,” Adv. Ther. (2009), 26(9), pp. 858-871 (published on line Sep. 4, 2009, printed Oct. 5, 2009) discloses compositions containing glucosamine sulfate and omega-3 fatty acids and their use in treating patients with osteoarthritis.

D'Altillio, “Therapeutic Efficacy and Safety of Undenatured Type II Collagen Singly or in Combination with Glucosamine and Chondroitin in Arthritic Dogs,” Toxicology Mechanisms and Methods, (2007), 17(4), pages 189-196 discloses the use of compositions containing glycosylated undenaturated Type II collagen in treating arthritis in dogs. The glycosylated undenatured Type II collagen is also used in combination with glucosamine HCl and chondroitin sulfate.

### BRIEF SUMMARY OF THE INVENTION

Provided in accordance with the present invention is a therapeutic composition comprising (1) at least one omega-3 polyunsaturated fatty acid, at least one pharmaceutically acceptable omega-3 polyunsaturated fatty acid derivative or mixtures thereof, (2) undenatured Type II collagen, and, optionally, (3) glucosamine sulfate wherein components (1), (2), and, when present, (3) are present in amounts effective to promote joint health in a subject.

In some embodiments, the therapeutic compositions may be compositions wherein the omega-3 polyunsaturated fatty acid derivatives are glycerides. In some embodiments, the omega-3 polyunsaturated fatty acid derivatives are derivatives of EPA and DHA.

Further provided in accordance with the present invention are compositions wherein component (1) is a mixture comprising about 35 wt. % triglycerides of EPA and about 25 wt. % triglycerides of DHA. As used herein, the term “about” means that the value or amount to which it refers can vary by ±5%.

The present invention further provides a therapeutic composition wherein component (1) is a mixture comprising at least about 60 wt. % of a combination of EPA and DHA in a weight ratio of EPA:DHA of from about 1.4:1 to about 5:1, wherein the combination is at least about 60% in the triglyceride form of the EPA and DHA and the balance is at least about 80% monoglycerides, diglycerides or both. Also provided are compositions wherein the combination comprises about 65 wt. % triglycerides of EPA and about 15 wt. % triglycerides of DHA or wherein the combination comprises about 75 wt. % triglycerides of EPA and about 15 wt. % triglycerides of DHA. Also provided are compositions wherein the combination is at least about 80% in the triglyceride form, at least about 90% in the triglyceride form, at least about 98% in the triglyceride form, or least about 98% in the triglyceride form and the remainder is monoglycerides, diglycerides or both. The present invention further provides therapeutic compositions wherein the combination comprises about 65 wt. % triglycerides of EPA and about 15 wt. % triglycerides of DHA.

The present invention also provides a dose of the medicament or therapeutic composition wherein the dose of medicament or therapeutic composition comprises about 200 mg to about 6 grams of EPA and/or derivatives of EPA plus DHA and/or derivatives of DHA, about 5 mg to about 100 mg of undenatured Type II collagen, and, when present, about 100 mg to about 3 grams of glucosamine sulfate. Typically, it takes about 40 mg of UC-II to provide about 10 mg undenatured Type II collagen.

Further provided in accordance with the present invention is a method of promoting joint health in a subject comprising administering to the subject a dosage comprising (1) at least one omega-3 polyunsaturated fatty acid, at least one pharmaceutically acceptable omega-3 polyunsaturated fatty acid derivative or mixtures thereof, (2) undenatured Type II collagen, and, optionally, (3) glucosamine sulfate wherein components (1), (2), and, when present, (3) are present in amounts effective to promote joint health in the subject.

### DETAILED DESCRIPTION OF THE INVENTION

In some embodiments, the therapeutic compositions of this invention include compositions derived from fish oil in which the fish oil comprises at least about 60% of omega-3 oils, or at least about 70% omega-3 oils. In some embodiments, the therapeutic compositions include compositions in which the omega-3 oils comprise about 50% EPA derivative and about 35% DHA derivative, or in which the omega-3 oils comprise about 69% EPA derivative and about 16% DHA derivative.

In some embodiments, the therapeutic compositions comprise a daily dose of the therapeutic compositions which is delivered by an integral number of capsules.

In some embodiments, the daily dose of medicament comprises about 200 mg to about 6 grams of EPA and/or derivatives of EPA plus DHA and/or derivatives of DHA, about 5 mg to about 100 mg of undenatured Type II collagen, and, when present, about 100 mg to about 3 grams of glucosamine sulfate. Typically, it takes about 40 mg of UC-II to provide about 10 mg undenatured Type II collagen. In some embodiments, the therapeutic composition further comprises an antioxidant. In some embodiments, the antioxidant is chosen from the group consisting of rosemary, vitamin E, astaxanthine, carnitine, and ascorbyl palmitate.

### Omega-3 Polyunsaturated Fatty Acids

As used herein, the term “omega-3 polyunsaturated fatty acid(s)” refers to a family of unsaturated fatty carboxylic acids that have in common a carbon-carbon double bond in the n-3 position (i.e., the third bond from the methyl end of the molecule). Typically, they contain from about 16 to about 24 carbon atoms and from three to six carbon-carbon double bonds. Omega-3 polyunsaturated fatty acids can be found in nature, and these natural omega-3 polyunsaturated fatty acids frequently have all of their carbon-carbon double bonds in the cis-configuration.

Examples of omega-3 polyunsaturated fatty acids include, but are not limited to, 7,10,13-hexadecatrienoic acid (sometimes abbreviated as 16:3 (n-3)); 9,12,15-octadecatetrienoic acid (a-linolenic acid (ALA), 18:3 (n-3)); 6,9,12,15-octadecatetraenoic acid (stearidonic acid (STD), 18:4 (n-3)); 11,14,17-eicosatrienoic acid (eicosatrienoic acid (ETE), 20:3 (n-3)); 8,11,14,17-eicosatetraenoic acid (eicosatetraenoic acid (ETA), 20:4 (n-3)); 5,8,11,14,17-eicosapentaenoic acid (eicosapentaenoic acid (EPA), (20:5 (n-3)); 7,10,13,16,19-docosapentaenoic acid (docosapentaenoic acid (DPA), 22:5 (n-3)); 4,7,10,13,16,19-docosahexaenoic acid (docosahexaenoic acid (DHA), 22:6 (n-3)); 9,12,15,18,21-tetracosapentaenoic acid (tetracosapentaenoic acid, 24:5 (n-3)); and 6,9,12,15,18,21-tetracosahexaenoic acid (tetracosahexaenoic acid, 24:6 (n-3)).

Eicosapentaenoic acid (EPA) and docosahexaenoic acid (DHA) are found in nature in fish oils and other natural sources, and have been used in a variety of dietary/therapeutic compositions. EPA and DHA are preferred omega-3 polyunsaturated fatty acids in the present invention.

The terms “EPA” and “DHA” are used herein in two contexts. When used in the context of an omega-3 polyunsaturated fatty acid, “EPA” and “DHA” refer to the free acid form of the omega-3 polyunsaturated fatty acid. When used in the context of omega-3 polyunsaturated fatty acid derivatives, “EPA” and “DHA” refer to the fact that the derivative contains an eicosapentaenoic acid moiety or docosahexaenoic acid moiety which is present as, for example, an ester, glyceride or phospholipid.

### Omega-3 Polyunsaturated Fatty Acid Derivatives

As used herein, the term “omega-3 polyunsaturated fatty acid derivative(s)” refers to omega-3 polyunsaturated fatty acids that have been reacted with another compound or otherwise modified so that the omega-3 polyunsaturated fatty acid no longer contains a free carboxylic acid. The derivative(s) may differ in activity (either greater or less) from its omega-3 polyunsaturated fatty acid parent, and may differ in activity from other derivatives. However, the derivative should retain sufficient activity to perform its intended function (in this case, promoting joint health). Examples of omega-3 polyunsaturated fatty acid derivatives include salts, esters (such as alkyl esters including, but not limited to, methyl and ethyl esters) and glycerides of omega-3 polyunsaturated fatty acids. The omega-3 polyunsaturated fatty acid can also be one or more of the fatty acid moieties in a phospholipid molecule.

As used herein, the term “glyceride” means a glycerol molecule (i.e., OHCH2CHOHCH2OH) in which one, two or all three of the hydroxyls have been esterified with a carboxylic acid, e.g., an omega-3 polyunsaturated fatty acid. Thus, “triglyceride” refers to glycerides in which all three hydroxyls on the glycerol have been esterified with (the same or different) carboxylic acids. “Diglyceride” refers to glycerides in which only two of the hydroxyls on the glycerol have been esterified with (the same or different) carboxylic acids. “Monoglyceride” refers to glycerides in which only one hydroxyl on the glycerol has been esterified with a carboxylic acid.

Omega-3 fatty acids can be found in nature in the triglyceride form (a glycerol with three fatty acids attached). The natural triglyceride form as found in raw fish oil cannot be readily separated as it occurs into purified EPA/DHA-containing mixtures by ordinary means such as distillation or crystallization, because the fatty acids are non-uniformly distributed among the triglyceride molecules. There are very few, if any, single triglyceride molecules which are composed of either three EPA moieties or three DHA moieties. Typically, there is a DHA moiety, an EPA moiety, and another fatty acid moiety in a triglyceride molecule. So in order to purify fatty acids to increase the proportion of EPA, DHA, or the total fraction of omega-3's, it is necessary to hydrolyze the triglycerides to remove at least some fatty acids from the glycerol.

The triglycerides may be converted by any method known to one skilled in the art without limitation. For example, the triglycerides may be converted by lipase-catalyzed esterification or lipase catalyzed acidolysis with ethyl or lauryl alcohol, which can selectively leave the highest amount of EPA and DHA bonded to glycerols and remove other components, leaving EPA and/or DHA as mono- or di-glycerides. The mono- and di-glycerides can then be separated into fractions with different EPA/DHA ratios, by methods familiar to those skilled in the art such as multiple stage vacuum distillation and/or fractional crystallization in urea. Advantageously, the purified EPA and DHA esters, after concentration, can be reattached to glycerol molecules using enzymatic reacylation to recreate glycerides which are otherwise identical to the original natural triglycerides, except that they are more concentrated in EPA and DHA combined, and they may also have a different ratio of EPA: DHA than the original fish oil. In some embodiments, at least 60% of the omega-3 fatty acids, and preferably 70% or more are converted to the triglyceride form in the reacylation process. The process may be successively repeated with addition of additional catalyst and/or enzyme and additional EPA and DHA until the desired specification proportions are met. About 60% of triglycerides can be made in the first pass of reacylation, with most of the remainder of the product being mono- and di-glycerides.

Polyunsaturated fatty acid triglycerides can be prepared using the following method.

### 1. Removal of Free Fatty Acids

Raw fish oil in the natural triglyceride molecular form preferably from anchovies and sardines which contain about 18% EPA and 12% DHA is heated to 60° C. to decrease viscosity. Sodium oxide is added to bind with free fatty acids in the oil. The mixture is moved to a separator where sodium oxide bound to free fatty acids (soap) floats to the top and is removed.

The oil is then moved to a second separator where warm water is preferably added to help remove traces of sodium oxide, as sodium oxide partitions to water, yet does not interact with the fish oil.

Citric acid may then be added to support splitting the oil from the combination of water and sodium oxide. The oil is then cooled to 30° C. to protect it from oxidation.

### 2. Stripping And Purification

Oil is moved to a separate stripping tank and heated to 200° C. Ethyl esters can be added to support the removal of impurities, which bind to ethyl esters. Impurities such as dioxins, heavy metals, polychlorinated biphenyls (PCBs), fire retardants, furans, and others evaporate and are drawn to the middle of the tank where a refrigerating element cools them down and drain them. The added esters are also removed with the impurities.

### 3. Esterification

The oil is moved to an esterification tank. Ethanol and sodium metal are added. Sodium metal is a catalyst for breaking off fatty acid strands from the glycerol backbone of the triglyceride fatty acid molecule, the free fatty acids then combined with ethanol to form ethyl esters. Water can be added to bind to sodium metal, where the combination of water and sodium metal can be removed.

### 4. Molecular Distillation

The oil is then moved to a distiller where it is heated to about 120° C. under vacuum. Mono esters and shorter carbon chain molecules move to the middle where they are cooled and drained, leaving longer carbon chains remaining as a concentrate. The process typically increases the key fatty acids by 100% during the first distillation; typically between 30-50% during the second distillation. The process can be repeated, although preferably the process is ideally only repeated once, as when oils undergo heat it can produce oxidation and degradation of the fatty acids in general. Oil waste is also increasing with repeated distillation, making the process less economical.

### 5. Reesterification (Reacylation)

The oil is then moved to a reesterification tank where the ethyl ester molecules are reconverted to the triglyceride form, which is the natural form of that fatty acid molecule. This natural triglyceride form comprises 98% of fats ingested by humans.

The esterification process takes place under low vacuum at about 80° C.

Glycerol is added to form the backbone of the glyceride molecules. Nitrogen can be added from the bottom of the tank to cause oil movement. Lipase enzymes are added as catalysts to facilitate the fatty acids binding to glycerol. The vacuum in the distillation tank removes the ethanol which was previously bound to the fatty acids. The enzymes used are lipases produced from bacteria or yeast. Perhaps the most effective enzymes are Candidan Antarctica lipase, and Chromobacterium Viscosum Lipase; other enzymes that can be used effectively are Psuedomonas, Mucor miehei, and Candida Cylindracea as well as other enzymes may also be used.

The reesterification process typically takes 24 hours, at which point the triglycerides typically reaches 60-65%, the remaining glycerides being diglycerides and monoglycerides. Around 3% of the fish oil will remain as ethyl esters, which can be removed together with the ethanol. Adding additional enzymes and/or continuing the enzymatic process can produce triglyceride molecule concentration of up to 99%. The 60-65% level is probably optimum from an economic point of view.

### 6. Winterization

The oil in triglyceride form is then moved to a cooling tank at 0° C., where saturated fats, in particular stearic acid are crystallized. The pulp is then pumped to a filter press, where the crystals are removed, essentially removing the vast majority of saturated fats from the oil. Depending on the amount of saturated fats in the oil, approximately 5-10% of the oil is lost during this process.

### 7. Bleaching

The oil is then removed to a bleaching tank at 60° C., where bleaching earth or bentonite earth is added to the oil. Any water in the oil evaporates due to the temperature. Any remaining impurities (trace minerals, etc) in the oil attach to the bentonite earth. The oil is then run through a bentonite earth filter to remove the bentonite earth together with the impurities.

### 8. Deodorization

Although not a necessary step, it is advantageous to move the oil to a deodorization tank. The tank contains low vacuum at 120° C. Steam is added at the bottom of the tank, which connects to color and odor molecules (oxidated matter, peroxides) which again travel into the vacuum system and into a residue container. This process gives the oil a neutral color with virtually zero taste and odor.

### 9. Mixing

The oil is then moved to a separate storage tank. Depending on the concentration of EPA and DHA desired, various batches can be mixed to yield the concentration desired for the final product.

### 10. Addition of Antioxidant

Antioxidants, in particular rosemary and mixed tocopherols can be added to the final oil to dramatically reduce the oxidation process.

### 11. Drumming

The oil is then drummed in stainless steel drums for storage and topped off with nitrogen to remove oxygen and minimize the potential for oxidation.

As used herein, the term “pharmaceutically acceptable” means that the material to which it refers is not harmful to the subject.

In some embodiments, the composition of the invention employs a mixture of omega-3 polyunsaturated fatty acids and/or derivatives that contain glycerides. For example, in one embodiment, the mixture contains about 35 wt. % triglycerides of EPA and about 25 wt. % triglycerides of DHA and about 10 wt. % other omega-3 fatty acids or derivatives thereof. In some embodiments, the mixture contains about 65 wt.% triglycerides of EPA, about 15 wt. % triglycerides of DHA and about 20 wt. % other omega-3 fatty acids or derivatives thereof, wherein the EPA and DHA are at least about 60% in the triglyceride form and the balance are at least about 90% of monoglycerides, diglycerides or both. In some embodiments, the mixture contains about 75 wt. % EPA and about 15 wt. % DHA, wherein at least about 60% of the combination of DHA and EPA are in the triglyceride form and the balance is at least about 90% monoglycerides, diglycerides or both. In another embodiment, the mixture can contain at least about 60 wt. % of a combination of EPA and DHA in a weight ratio of EPA:DHA of from about 1.4:1 to about 5:1 (for example, 2:1 to 5:1, 3:1, 4:1 or 4.3:1) wherein the combination is at least about 60% (e.g., at least about 80% or at least about 90% or at least about 98%) in the triglyceride form of the fatty acids and the balance is at least about 80% monoglycerides, diglycerides or both. In some embodiments, the combination is at least about 98% in the triglyceride form, with the balance being in the monoglyceride and/or diglyceride forms. Some of the above compositions are disclosed in copending U.S. patent application Ser. No. 12/015,488, filed Jan. 16, 2008 by Opheim. That patent application is incorporated by reference herein in its entirety.

Sources of the omega-3 polyunsaturated fatty acids or derivatives thereof include natural sources including, but not limited to, fish oil (e.g., cod liver oil), flax seed oil, marine oils, sea oils, krill oil, algae and the like. Fish oil is a preferred source.

It is preferred to use a high quality source of omega-3 polyunsaturated fatty acids or derivatives thereof which is rich in omega-3 oils, preferably containing at least 70% omega-3 oils. The oil can also be rich in EPA and DHA moieties. Preferably, at least 75% of the omega oils contain EPA+DHA moieties, and more preferably 85% or more contain EPA+DHA moieties. The daily dose of omega-3 oils is about 1 to about 4 grams of omega-3 oil. One possible source is a balanced omega-3 formula such as Nordic Naturals, Inc.'s ProOmega nutritional supplement, which is 70% omega-3 oils of which 50.8% EPA moieties, 35.1% contains DHA moieties and 14.1% is other omega-3 polyunsaturated fatty acids or derivatives thereof.

One preferred source of omega-3 polyunsaturated fatty acids or derivatives thereof is Pro-EPA nutritional supplement sold by Nordic Naturals, Inc. It comprises 69.1% EPA derivative, 16.3% DHA derivative, and 14.6% other omega-3 polyunsaturated fatty acids or derivatives thereof. Still another preferred source of omeg-3 polyunsaturated fatty acids or derivatives thereof is Nordic Naturals, Inc.'s Pro-EFA Xtra which comprises 56.9% EPA derivative, 14.7% DHA derivative, 17.2% GLA (omega-6 gamma-linolenic acid, i.e., 6,9,12-octadecatrienoic acid (18:3 (n-6) or derivative thereof), and 11.2% other omega-3 polyunsaturated fatty acids or derivatives thereof. The Pro-EFA Xtra formula adds an omega-6 polyunsaturated fatty acid or derivatives thereof, GLA, and makes a powerful anti-inflammatory mixture.

In some embodiments, component (1) of the therapeutic compositions of the present invention contains polyunsaturated fatty acids or derivatives thereof other than omega-3 polyunsaturated fatty acids or derivatives thereof. For example, component (1) can contain omega-6 and/or omega-9 polyunsaturated fatty acids or derivatives thereof (e.g., esters, glycerides or phospholipids).

### Glucosamine Sulfate

The compositions of the present invention can optionally contain glucosamine sulfate. Glucosamine sulfate is a known compound. It is produced commercially by the hydrolysis of crustacean exoskeletons, or by fermentation of a grain such as corn or wheat. When used, a typical dose of the compositions of the invention contains about 100 mg to about 3 grams of glucosamine sulfate.

### Undenatured Type II Collagen

Undenatured Type II collagen (sometimes referred to as “UC-II”) and methods for producing it are disclosed in aforementioned U.S. Pat. Nos. 5,529,786; 5,637,321; 5,645,851; and 5,750,144.

Undenatured Type II collagen can be obtained from animal tissue, such as chicken cartilage from chickens less than about one year old. Other animal tissue containing Type II collagen, such as bovine cartilage and the vitreous humor of eyes, may also be used as a source of undenatured Type II collagen. In the case of chickens, the cartilage is dissected free of surrounding tissues and diced or comminuted. The diced cartilage is sterilized by known techniques and formed into a size and shape suitable for administration to a subject (such as in the form of a capsule).

UC-II is available commercially as UC-250 powder from InterHealth Nutraceuticals Incorporated.

The compositions of this invention can contain other ingredients besides the ingredients recited above. These include, but are not limited to, flavor agents, fillers, surfactants (e.g., polysorbate 80 and sodium lauryl sulfate), color agents including, e.g., dyes and pigments, sweeteners, antioxidants and additional ingredients, such as vitamins, minerals and herbs.

### Flavor Agents

Useful flavor agents include natural and synthetic flavoring sources including, but not limited to, volatile oils, synthetic flavor oils, flavoring aromatics, oils, liquids, oleoresins, and extracts derived from plants, leaves, flowers, fruits, stems, and combinations thereof. Useful flavor agents include, e.g., citric oils, e.g., lemon, orange, grape, lime, and grapefruit, fruit essences including, e.g., apple, pear, peach, banana, grape, berry, strawberry, raspberry, blueberry, blackberry, cherry, plum, pineapple, apricot, and other fruit flavors. Other useful flavor agents include, e.g., aldehydes and esters (e.g., benzaldehyde (cherry, almond)), citral, i.e., alpha-citral (lemon, lime), neral, i.e., beta-citral (lemon, lime), decanal (orange, lemon), aldehyde C-8 (citrus fruits), aldehyde C-9 (citrus fruits), aldehyde C-12 (citrus fruits), tolyl aldehyde (cherry, almond), 2,6-dimethyloctanal (green fruit), 2-dodedenal (citrus, mandarin), and mixtures thereof, chocolate, cocoa, almond, cashew, macadamia nut, coconut, mint, chili pepper, pepper, cinnamon, vanilla, tooty fruity, mango, and green tea. Mixtures of two or more flavor agents may also be employed. When a flavor agent is used, the amount employed will depend upon the particular flavor agent used. However, in general, the flavor agent can constitute from about 5% to about 50% by weight of the composition.

### Color Agents

Useful color agents include, e.g., food, drug, and cosmetic (FD&C) colors including, e.g., dyes, lakes, and certain natural and derived colorants. Useful lakes include dyes absorbed on aluminum hydroxide and other suitable carriers. Mixtures of color agents may also be employed. When a color agent is employed, the amount used will depend upon the particular color agent used; however, in general, the color agent can constitute from about 0.5% to about 5% by weight of the composition.

### Sweetening Agent

Natural and/or artificial sweetening agents can also be added to the composition. Examples of sweeteners include sugars such as sucrose, glucose, invert sugar, fructose, and mixtures thereof, saccharin and its various salts (e.g., sodium and calcium salt of saccharin), cyclamic acid and its various salts, dipeptide sweeteners (e.g., aspartame), dihydrochalcone, and sugar alcohols including, e.g., sorbitol, sorbitol syrup, mannitol and xylitol, and combinations thereof. Natural sweeteners that can be employed include, but are not limited to, luo han, stevia or mixtures thereof. Luo han sweetener is derived from luo han guo fruit (siraitia grosvenorii) that is mainly found in China. It is about 300 times sweeter by weight than sucrose. Luo han is commercially available from, e.g., Barrington Nutritionals (Harrison, N.Y.). Stevia is derived from a South American herb, Stevia rebaudiana. It can be up to about 300 times sweeter than sucrose. Because luo han and stevia have such a sweet taste, only a small amount need be used in the composition. When a sweetening agent is employed the amount used will depend upon the particular sweetening agent used; however, in general, the sweetening agent can constitute from about 0.0005% to about 30%, by weight of the composition. When a sweetener having a very sweet taste, such as luo han or stevia, is used, small amounts such as about 0.0005% to about 0.1% (for example about 0.005% to about 0.015% or about 0.002% to about 0.003%) by weight can be used.

### Additional Ingredients

The compositions of the present invention can contain additional ingredients. Examples of such additional ingredients include, but are not limited to, vitamins, minerals, and/or herbs.

As used herein, the term “vitamin” refers to trace organic substances that are required in the diet. For the purposes of the present invention, the term vitamin(s) include, without limitation, thiamin, riboflavin, nicotinic acid, pantothenic acid, pyridoxine, biotin, folic acid, vitamin B 12, lipoic acid, ascorbic acid, vitamin A, vitamin D, vitamin E, and vitamin K. Also included within the term vitamin are the coenzymes thereof. Coenzymes are specific chemical forms of vitamins. Coenzymes include thiamine pyrophosphates (TPP), flavin mononucleotide (FMM), flavin adenine dinucleotive (FAD), Nicotinamide adenine dinucleotide (AND), Nicotinamide adenine dinucleotide phosphate (NADP), Coenzyme A (CoA), Coenzyme Q10 (CoQ 10), pyridoxal phosphate, biocytin, tetrahydrofolic acid, coenzyme B 12, lipoyllysine, 11-cis-retinal, and 1,25-dihydroxycholecalciferol. The term vitamin(s) also includes choline, camitine, and alpha, beta, and gamma carotenes.

As used herein, the term “mineral” refers to inorganic substances, metals, and the like required in the human diet. Thus, the term “mineral” as used herein includes, without limitation, calcium, iron, zinc, selenium, copper, iodine, magnesium, phosphorus, chromium and the like, and mixtures thereof. Compounds containing these elements are also included in the term “mineral.”

As used herein, the term “herb” refers to organic substances defined as any of various often aromatic plants used especially in medicine or as seasoning. Thus, the term “herb” as used herein includes, but is not limited to, black currant, ginsing, ginko bilboa, cinnamon, and the like, and mixtures thereof.

In some embodiments, a dosage of the therapeutic compositions further includes antioxidants such as rosemary, vitamin E, astaxanthine, carnitine, and ascorbyl palmitate or other antioxidants known in the art for stabilizing fish oil and/or omega-3 polyunsaturated fatty acids or derivatives thereof. When used, these antioxidants are employed in an amount sufficient to deter or prevent oxidation of the fish oil and/or omega-3 polyunsaturated fatty acids or derivatives thereof.

The compositions of this invention are suitable for therapeutic and/or nutritional purposes in treating a subject in need of such treatment. As used herein, the term “subject” includes, but is not limited to, a non-human animal, such as a cow, monkey, horse, sheep, pig, chicken, turkey, quail, cat, dog, mouse, rat, rabbit, or guinea pig; and a human. Typically, the subject is a mammal, most typically a human.

The amount of the composition of the invention that is effective will vary depending upon the condition being treated, and can be determined by standard clinical techniques. The precise dose to be employed will also depend on the relative amounts of the components of the compositions of the invention, route of administration, and the seriousness of the condition being treated and should be decided according to the judgment of the practitioner and each subject's circumstances. However, suitable effective dosage amounts for the compositions of the invention typically are 200 mg to about 6 grams of EPA and/or derivatives of EPA plus DHA and/or derivatives of DHA and about 5 mg to about 100 mg of undenatured Type II collagen. When used, a typical dose of the compositions of the invention contains about 100 mg to about 3 grams of glucosamine sulfate. Typically, it takes about 40 mg of UC-II to provide about 10 mg undenatured Type II collagen.

The compositions of the present invention comprise (1) at least one omega-3 polyunsaturated fatty acid, or at least one pharmaceutically acceptable omega-3 polyunsaturated fatty acid derivative or mixtures thereof, (2) undenatured Type II collagen and, optionally, (3) glucosamine sulfate wherein components (1), (2), and, when present, (3) are present in amounts effective to promote joint health in a subject. The phrase “present in amounts effective to promote joint health in a subject” as used herein means that components (1), (2), and (3) are used in an amount, individually and in combination, effective for a therapeutic, preventive or nutritional activity in a subject that promotes joint help in the subject. By “promote joint health in a subject” is meant the compositions helps improve (or at least helps maintain) the health of the subject's joints, and especially the cartilage in the subject's joints. By “amount individually and in combination effective” is meant that each individual component is present in an amount sufficient to perform its function as well as the overall composition being in an amount sufficient to perform its overall function.

The form in which the composition of the invention is administered to the subject is not critical. Typically, the composition is administered as a liquid or in a capsule. Typically, the composition is administered in the form of individual doses. As used herein, the term “dose” includes both the case where components (1), (2), and, when present, (3) are administered together (such as in the form of a capsule containing all three components), and the case where components (1), (2), and, when present, (3) are administered separately (but, typically, at essentially the same time). In some embodiments, the composition of the invention is administered in the form of a daily dose. However, depending on the severity of the condition being treated, this may not be required, and the period between administration of the doses may be longer than one day. In addition, the term “administer” includes both the case where a third party administers the dose to the subject and the case where the subject self-administers the dose.

The compositions of the present invention promote joint health in a subject. They can be administered to a subject with a connective tissue disorder to help build cartilage and significantly improve the disorder. The compositions can be used to treat, for example, degenerative joint diseases (i.e., rheumatoid arthritis), joint defects, osteoarthritis, polychondritis, vascular disease, cartilage injuries, autoimmune diseases involving connective tissue autoantibodies (rheumatoid arthritis) and any other connective tissue disorder that would benefit from increased synthesis of cartilage.

### EXAMPLES

A composition according to the present invention is prepared using the following ingredients in the amounts shown below. A single serving of the composition (in the form of three soft gel capsules) contains:

| Ingredient | Amount | |
|---|---|---|
| Fish oil (35 wt. % EPA glycerides, 25 wt. % | 2000 | mg |
| DHA glycerides, other omega-3 fatty acid | | |
| derivatives, omega-9 fatty acid derivative) | | |
| Vitamin D3 (cholecalciferol) | 1000 | IU |
| Vitamin E (d-alpha tocopherols) | 30 | IU |
| UC-II (providing 10 mg of undenatured | 40 | mg |
| Type II collagen) | | |
| EPA glycerides | 700 | mg |
| DHA glycerides | 500 | mg |
| Total omega-3s | 1200 | mg. |

Other ingredients include rosemary extract (lipid stabilizer antioxidant), yellow beeswax (to suspend ingredients in the capsules), and coloring agent. The composition can optionally contain 1500 mg of glucosamine sulfate (derived from fermented corn source).

Although the present invention has been described in considerable detail with reference to certain versions thereof, other versions are possible. Therefore the spirit and scope of the appended claims should not be limited to the versions presented herein.

What is claimed is:

1. A therapeutic composition comprising (1) at least one omega-3 polyunsaturated fatty acid, at least one pharmaceutically acceptable omega-3 polyunsaturated fatty acid derivative or mixtures thereof, and (2) undenatured Type H collagen wherein components (1) and (2) are present in amounts effective to promote joint health in a subject.

2. The therapeutic composition of claim 1 wherein component (1) comprises EPA, derivatives of EPA, DHA, derivatives of DHA or mixtures thereof.

3. The therapeutic composition of claim 2 wherein component (1) comprises a derivative of EPA.

4. The therapeutic composition of claim 2 wherein component (1) comprises a derivative of DHA.

5. The therapeutic composition of claim 2 wherein component (1) comprises a mixture of a derivative of EPA and a derivative of DHA.

6. The therapeutic composition of claim 2 wherein the derivatives of EPA and derivatives of DHA are selected from the group consisting of alkyl esters, glycerides and phospholipids and mixtures thereof.

7. The therapeutic composition of claim 6 wherein the derivatives of EPA and derivatives of DHA are glycerides.

8. The therapeutic composition of claim 1 wherein component (1) is a mixture comprising about 35 wt. % triglycerides of EPA and about 25 wt. % triglycerides of DHA.

9. The therapeutic composition of claim 1 wherein component (1) is a mixture comprising at least about 60 wt. % of a combination of EPA and DHA in a weight ratio of EPA:DHA of from about 1.4:1 to about 5:1, wherein the combination is at least about 60% in the triglyceride form of the EPA and DHA and the balance is at least about 80% monoglycerides, diglycerides or both.

10. The therapeutic composition of claim 9 wherein the combination is at least about 80% in the triglyceride form.

11. The therapeutic composition of claim 9 wherein the combination is at least about 90% in the triglyceride form.

12. The therapeutic composition of claim 9 wherein the combination is at least about 98% in the triglyceride form.

13. The therapeutic composition of claim 12 wherein the combination is at least about 98% in the triglyceride form and the remainder is monoglycerides, diglycerides or both.

14. The therapeutic composition of claim 9 wherein the combination comprises about 65 wt. % triglycerides of EPA and about 15 wt. % triglycerides of DHA.

15. The therapeutic composition of claim 14 wherein the combination is at least about 80% in the triglyceride form.

16. The therapeutic composition of claim 14 wherein the combination is at least about 90% in the triglyceride form.

17. The therapeutic composition of claim 14 wherein the combination is at least about 98% in the triglyceride form.

18. The therapeutic composition of claim 17 wherein the combination is at least about 98% in the triglyceride form and the remainder is monoglycerides, diglycerides or both.

19. The therapeutic composition of claim 9 wherein the combination comprises about 75 wt. % triglycerides of EPA and about 15 wt. % triglycerides of DHA.

20. The therapeutic composition of claim 19 wherein the combination is at least about 80% in the triglyceride form.

21. The therapeutic composition of claim 19 wherein the combination is at least about 90% in the triglyceride form.

22. The therapeutic composition of claim 19 wherein the combination is at least about 98% in the triglyceride form.

23. The therapeutic composition of claim 22 wherein the combination is at least about 98% in the triglyceride form and the remainder is monoglycerides, diglycerides or both.

24. The therapeutic composition of claim 1 further comprising a soft gelatin capsule into which components (1) and (2) are loaded.

25. The therapeutic composition of claim 24 wherein a daily dose of the therapeutic composition is delivered by an integral number of capsules.

26. The therapeutic composition of claim 1 wherein a daily dose of the therapeutic composition comprises about 200 mg to about 6 grams of EPA and/or derivatives of EPA plus DHA and/or derivatives of DHA, and about 5 mg to about 100 mg of undenatured Type II collagen.

27. The therapeutic composition of claim 1 further comprising an antioxidant.

28. The therapeutic composition of 27 wherein the antioxidant is chosen from the group consisting of rosemary, vitamin E, astaxanthine, carnitine, and ascorbyl palmitate.

29. The therapeutic composition of claim 1 further comprising Vitamin D.

30. A method of promoting joint health in a subject comprising administering to the subject a dosage comprising (1) at least one omega-3 polyunsaturated fatty acid, at least one pharmaceutically acceptable omega-3 polyunsaturated fatty acid derivative or mixtures thereof and (2) undenatured Type II collagen wherein components (1) and (2) are present in amounts effective to promote joint health in the subject.

31. The method of claim 30 wherein component (1) comprises EPA, derivatives of EPA, DHA, derivatives of DHA or mixtures thereof.

32. The therapeutic composition of claim 31 wherein component (1) comprises a derivative of EPA.

33. The therapeutic composition of claim 31 wherein component (1) comprises a derivative of DHA.

34. The therapeutic composition of claim 31 wherein component (1) comprises a mixture of a derivative of EPA and a derivative of DHA.

35. The method of claim 30 wherein the derivative of EPA and derivative of DHA are selected from the group consisting of alkyl esters, glycerides, phospholipids and mixtures thereof.

36. The method of claim 35 wherein the derivative of EPA and derivative of DHA are glycerides.

37. The method of claim 30 wherein component (1) is a mixture comprising about 35 wt. % triglycerides of EPA and about 25 wt. % triglycerides of DHA.

38. The method of claim 30 wherein component (1) is a mixture comprising at least about 60 wt. % of a combination of EPA and DHA in a weight ratio of EPA:DHA of from about 1.4:1 to about 5:1, wherein the combination is at least about 60% in the triglyceride form of the fatty acids and the balance is at least about 80% monoglycerides, diglycerides or both.

39. The method of claim 38 wherein the combination is at least about 80% in the triglyceride form.

40. The method of claim 38 wherein the combination is at least about 90% in the triglyceride form.

41. The method of claim 38 wherein the combination is at least about 98% in the triglyceride form.

42. The method of claim 41 wherein the combination is at least about 98% in the triglyceride form and the remainder is monoglycerides, diglycerides or both.

43. The method of claim 38 wherein the combination comprises about 65 wt. % triglycerides of EPA and about 15 wt. % triglycerides of DHA.

44. The method of claim 43 wherein the combination is at least about 80% in the triglyceride form.

45. The method of claim 43 wherein the combination is at least about 90% in the triglyceride form.

46. The method of claim 43 wherein the combination is at least about 98% in the triglyceride form.

47. The method of claim 46 wherein the combination is at least about 98% in the triglyceride form and the remainder is monoglycerides, diglycerides or both.

48. The method of claim 38 wherein the combination comprises about 75 wt. % triglycerides of EPA and about 15 wt. % triglycerides of DHA.

49. The method of claim 48 wherein the combination is at least about 80% in the triglyceride form.

50. The method of claim 48 wherein the combination is at least about 90% in the triglyceride form.

51. The method of claim 48 wherein the combination is at least about 98% in the triglyceride form.

52. The method of claim 51 wherein the combination is at least about 98% in the triglyceride form and the remainder is monoglycerides, diglycerides or both.

53. The method of claim 30 further comprising a soft gelatin capsule into which components (1) and (2) are loaded.

54. The method of claim 53 wherein a daily dose of the therapeutic composition is delivered by an integral number of capsules.

55. The method of claim 30 wherein a daily dose of the therapeutic composition comprises about 200 mg to about 6 grams of EPA and/or derivatives of EPA plus DHA and/or derivatives of DHA, and about 5 mg to about 100 mg of undenatured Type II collagen.

56. The method of claim 30 wherein the dosage further comprises an antioxidant.

57. The method of claim 56 wherein the antioxidant is chosen from the group consisting of rosemary, vitamin E, astaxanthine, carnitine, and ascorbyl palmitate.

58. The method of claim 30 wherein the dosage further comprises Vitamin D.
